(19) United States
(12) Patent Application Publication
(10) Pub. No.: US 2025/0319467 A1
(43) Pub. Date: Oct. 16, 2025

(54) CELL ISOLATION AND REAGENT EXCHANGE IN A MICROFLUIDIC DEVICE

(71) Applicant: MAYO FOUNDATION FOR MEDICAL EDUCATION AND RESEARCH, Rochester, MN (US)

(72) Inventors: Kevin D. Loutherback, Rochester, MN (US); Allan B. Dietz, Rochester, MN (US)

(21) Appl. No.: 18/698,764

(22) PCT Filed: Oct. 5, 2022
(86) PCT No.: PCT/US2022/045762

§ 371 Date: Apr. 4, 2024

Related U.S. Application Data
(60) Provisional application No. 63/252,471, filed on Oct. 5, 2021.

(51) Int. Cl. B01L 3/00; G01N 33/533

(52) U.S. Cl. CPC B01L 3/502761; G01N 33/533; B01L 2200/027; B01L 2200/0652; B01L 2300/0809; B01L 2300/0848; B01L 2300/0893; B01L 2400/043

(57) ABSTRACT

A microfluidic device includes a channel layer configured for flow of a fluid medium; and a plurality of microwells in fluid communication with the channel layer, the plurality of microwells each comprising a well depth and a well diameter that prevents fluid flow of the fluid medium into the plurality of microwells during circulation of the fluid medium in the channel layer.

### CLAIM OF PRIORITY

This application is a National Stage application under 35 U.S.C. § 371 of International Application No. PCT/US2022/045762, having an International Filing Date of Oct. 5, 2022, which claims priority under 35 U.S.C. § 119 (e) to U.S. Patent Application Ser. No. 63/252,471, filed on Oct. 5, 2021, the entire contents of which are hereby incorporated by reference.

### TECHNICAL FIELD

This disclosure generally relates to microfluidic devices.

### BACKGROUND

A microfluidic system can include a single tissue construct or an interconnected set of two-dimensional (2D) or three-dimensional (3D) cellular constructs that are frequently referred to as organs-on-chips, tissue chips, or in vitro organ constructs. The constructs are typically made with immortalized cell lines, primary cells from animals or humans, or organ-specific cells derived from naïve cells, human embryonic stem cells, and induced pluripotent stem cells (iPSCs). Individually, each construct can be designed to recapitulate the structure and function of a human organ or organ region, paying particular attention to the cellular microenvironment and cellular heterogeneity. When coupled together, these constructs offer the possibility of providing, in vitro, an unprecedented physiological accuracy for the study of cell-cell, drug-cell, drug-drug, and organ-drug interactions, if drug dynamics (e.g., drug interaction with tissue) can be properly modeled.

### SUMMARY

This specification describes systems and processes for a microfluidic microwell device that allows cell isolation and reagent exchange to be integrated in a single device. A microfluidic device is composed of an open channel with microwells patterned along an upper wall of the device. The microwell and channel geometry enable immunomagnetic isolation of cells labeled with antibody-conjugated magnetic nanoparticles from unlabeled cells. The microfluidic device enables these labeled cells to be introduced (e.g., from an external source) into the channel. The cells can be pulled into one or more microwells by a magnet. The unlabeled cells remain in the main channel or in sediment due to gravity. Generally, there is no flow from the channel into the wells. Thus, only cells labeled with the magnetic nanoparticles are captured in the microwells. Unlabeled cells are flushed from the microfluidic device (e.g., after cell separation).

The microfluidic device enables generation of mature dendritic cells from various cell types. For example, mature dendritic cells can be captured from CD14+ monocytes and other cell types. The mature dendritic cells can be used for dendritic cell (DC) therapies, which are used to treat for diseases such as cancers. The microfluidic device including the microfluidic microwells is therefore configured for each of monocyte isolation, differentiation, and maturation in a single device. In the microfluidic device, cell isolation includes separation of one target population of cells from a larger population that includes a mixture of the target and non-target cells. Cell differentiation includes a biological process of cells changing from one cell type to another cell type within the microfluidic device. Cell differentiation is performed in the microfluidic device by culturing cells with a mixture of cytokines that cause the cultured cells to change their states.

Implementations of the present disclosure can provide one or more of the following advantages. The microfluidic device can enable immunomagnetic isolation of cells in microwells of the microfluidic system in a manner with low shear stress and in which cell loss is minimized. The separation technique uses magnetic beads conjugated to an antibody against a specific cell surface marker to isolate cells expressing that marker within a mixture of cells. When placed in a magnetic field, labeled cells are drawn away from other cells and collected. The geometry of the microwells includes a depth that prevents flow from sweeping out cells within the well. These microwells provide the low shear stress environment for cell culture and reduce cell loss while still maintaining a fluidic connection to the channel to allow fluid exchange. After cell capture, the microfluidic device can be flipped such that gravity maintains the captured cells in the microwells. Fluid in the main channel is changed to allow reagent exchange such as in situ labeling with fluorescent markers, washing, and enumeration and characterization.

Additionally, the microfluidic device is capable of integrating the steps of the dendritic cell manufacturing process onto a single device. The microfluidic device enables cell isolation surface marker characterization and development of a culture of mature dendritic cells. The microfluidic device is scalable. The microfluidic device can be patterned over a large area to accommodate millions of cells, manufactured in plastic using high volume manufacturing techniques and operated in a standard lab environment rather than a GMP space, because the microwells are completely closed. The process for culturing cells in the microfluidic device is transferrable to other cell therapy manufacturing protocols including CAR-T, TIL, ECT, CAR-NK, and so forth.

These advantages are realized by one or more of the following embodiments.

In a general aspect, a microfluidic device includes a channel layer configured for flow of a fluid medium; and a plurality of microwells in fluid communication with the channel layer, the plurality of microwells each comprising a well depth and a well diameter that prevents fluid flow of the fluid medium into the plurality of microwells during circulation of the fluid medium in the channel layer.

In some implementations, the well depth and the well diameter of each of the plurality of microwells are each determined based on an expected flow rate of the fluid medium in the channel layer.

In some implementations, for a given microwell, the well depth and the well diameter of the given microwell together cause fluid medium present in the given microwell to form one or more vortices during circulation of the fluid medium in the fluid channel, wherein the one or more vortices prevent the fluid medium from flowing into the given microwell from the channel layer.

In some implementations, the well depth is 100 micrometers, wherein a flow rate of the fluid medium is between 1 micrometer per second and 10 centimeters per second, and wherein the well diameter is one of: 10-30 micrometers causing three or more vortices in the given microwell during the circulation of the fluid medium; 40-50 micrometers causing two vortices in the given microwell during the circulation of the fluid medium; or 60-100 micrometers causing one vortex in the given microwell during the circulation of the fluid medium.

In some implementations, the microfluidic device includes a magnet configured to pull cells labeled with magnetic nanoparticles into the plurality of microwells during a cell separation process.

In some implementations, the channel layer is 200 micrometers thick.

In some implementations, the well depth is approximately 100 micrometers.

In some implementations, the well diameter is between 10-100 micrometers.

In some implementations, the channel layer and microwells are formed from polydimethylsiloxane (PDMS).

In a general aspect, a method of labeling cells with a microfluidic device, the method comprising: introducing a fluid medium into a channel and microwells of the microfluidic device, the plurality of microwells being in fluid communication with the channel; orienting the microwells of the microfluidic device to be above a channel of the microfluidic device; introducing cells into the fluid medium, wherein a first portion of the cells are labeled with magnetic particles, and wherein a second portion of the cells are not labeled with the magnetic particles; applying a magnetic force to the microfluidic device to pull the first portion of the cells into the microwells, wherein the second portion of the cells remain in the channel; circulating the fluid medium in the channel to remove the second portion of the cells, wherein a geometry of the microwells prevents flow of the fluid medium from the channel into the microwells. Here, preventing flow includes substantially or completely preventing flow such that less than 10%, 5%, 2%, 1%, or 0.5% of fluid volume flowing through the channel is introduced into the microwells. In some implementations, the flow prevention is less than 0.5% for wells having a depth that is ten times the diameter of the well or greater.

In some implementations, the geometry of the microwells that prevents flow of the fluid medium from the channel into the microwells is determined based on an expected flow rate of the fluid medium.

In some implementations, the flow rate is between 1 micrometer per second and 10 centimeters per second.

In some implementations, the geometry of the microwells that prevents flow of the fluid medium from the channel into the microwells comprises a microwell width and a microwell depth.

In some implementations, the microwell width is 30 micrometers, wherein the well depth is 100 micrometers, and wherein the channel depth is 200 micrometers.

In some implementations, the geometry of the microwells that prevents flow of the fluid medium from the channel into the microwells causes one or more vortices to form from fluid medium in at least one of the microwells during circulation of the fluid medium, the one or more vortices preventing fluid medium from flowing from the channel into the at least one microwell.

In some implementations, the method includes performing cell labeling of the first portion of the cells by circulating a second fluid medium including fluorophore conjugated antibodies into the channel and allowing the fluorophore conjugated antibodies to diffuse into the microwells.

In a general aspect, a method of forming a microfluidic device, the method comprising: obtaining a first layer of polydimethylsiloxane (PDMS); forming a channel in the first layer of PDMS; obtaining a second layer of polydimethylsiloxane (PDMS); forming a plurality of microwells in the second layer of PDMS by photolithography, the plurality of microwells having a well diameter and a well depth configured to prevent fluid flow of the fluid medium into the plurality of microwells during circulation of the fluid medium in the channel layer; bonding the first layer of PDMS to the second layer of PDMS; and forming fluid connections between the channel and the plurality of microwells.

In some implementations, the fluid connections are formed by holes punched in the first layer. These and other aspects, features, and implementations will become apparent from the following descriptions, including the claims.

### BRIEF DESCRIPTION OF THE DRAWINGS

FIGS. 1A-1C each is a portion of a flow diagram illustrating an example process.

FIG. 2 is a flow diagram illustrating an example process.

FIG. 3A shows an image of a microfluidic device.

FIG. 3B shows an image of a microfluidic device.

FIG. 3C shows an image of a microfluidic device.

FIG. 3D shows an image of a microfluidic device.

FIG. 4 is a graph illustrating cell retention.

FIG. 5A is a diagram showing a simulation of fluid velocity in a device.

FIG. 5B is a graph showing simulation results for the device of FIG. 5A.

FIG. 6A shows simulations of fluid velocity.

FIG. 6B shows simulations of fluid velocity.

FIG. 6C shows simulations of fluid velocities.

FIG. 6D shows a graph of fluid velocity reduction during a cell sweep process for microwells of FIG. 6C.

FIG. 7A shows images of a cell marking process using the microwells of FIGS. 1-6D.

FIG. 7B shows scatterplots.

FIG. 8 shows a flow diagram showing a process for dendritic cell (DC) isolation.

FIG. 9 includes scatterplots.

FIG. 10 shows an example of DC isolation.

FIG. 11 shows a flow diagram of a process for cell isolation.

FIG. 12 shows a flow diagram of a process for forming a microfluidic device.

### DETAILED DESCRIPTION

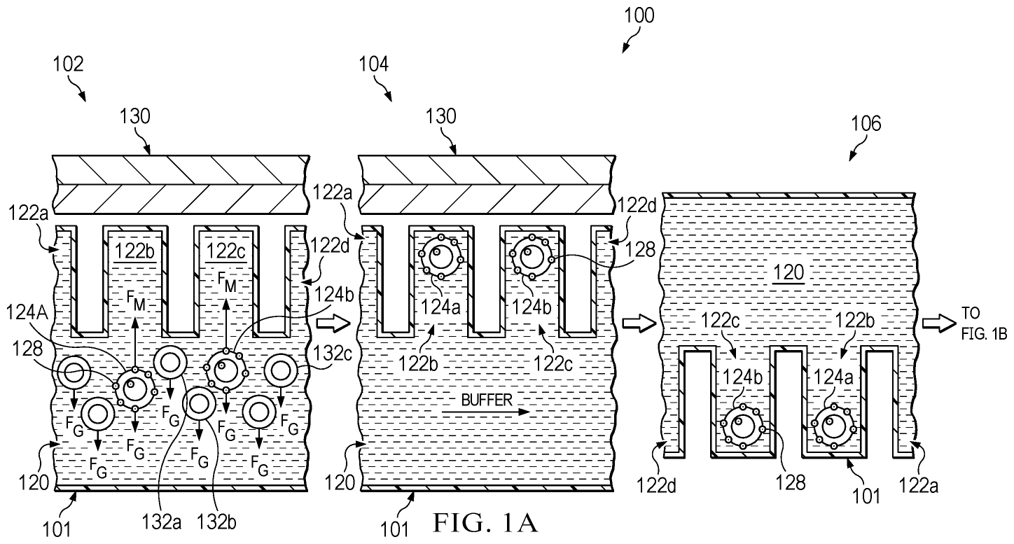

FIGS. 1A-1C each shows a portion of a flow diagram illustrating an example process 100 for cell isolation and reagent exchange in a microfluidic device, such as an on-chip microfluidic device. In FIG. 1A, the microfluidic device 101 is shown from a side-view. The microfluidic device 101 includes an open channel 120 (also called a main channel). The open channel allows fluid to flow through the microfluidic device 101 and across microwells 122. The fluid is input from an external source into the microfluidic device 101. Microwells 122a-d (also called wells) are patterned on a wall (e.g., an upper wall) of the channel 120. Cells 124a-b are labeled with antibody-conjugated magnetic nanoparticles 128. A magnet 130 is positioned vertically above the microwells. The labeled cells 124a-b and unlabeled cells 132a-c are introduced into the channel 120 at step 102. The cells are introduced in a flowing liquid that is input into the microfluidic device 101. The microwells 122a-d are shaped so that there is no flow (e.g., substantially no flow) from the channel 120 into the wells 122a-d.

For introducing cells into the microwells, the fluid flow through the channel 120 is stopped. The cells 124a-b labeled with the magnetic nanoparticles 128 are suspended in the fluid media. A magnet 130 is introduce to apply a magnetic force F_{M} to the labeled cells 124. The labeled cells 124 are pulled into the microwells 122a-d by a magnetic force F_{M} exerted by the magnet 130 on the antibody-conjugated magnetic nanoparticles 128 that label the cells 124a-b. Unlabeled cells 132a-c remain in the main channel 120 or sediment under an influence of gravity (depicted as gravitational force F_{G}).

Once the cells 124 are in the microwells 122, the flow resumes. The unlabeled cells 132 are flushed from the microfluidic device at step 104, e.g., by flowing a buffer solution through the channel 120. The microfluidic device is flipped at step 106 so that gravity retains the captured cells 124a-b in the microwells.

As shown in FIG. 1B, the fluid in the main channel can be changed while the captured cells 124a-b are retained in the wells 122a-d by flowing a replacement fluid through the channel 120. The fluid can be changed to allow reagent exchange. As shown at step 108, the fluid can be replaced with reagent such as for in situ labeling of the cells 124 with fluorescent markers 126, washing, enumeration, characterization, or a combination thereof. As shown at step 110, in some implementations, on-chip labeling is performed by flowing a solution containing the fluorescent markers 126, e.g., fluorophore conjugated antibodies, through the channel and allowing the fluorophore conjugated antibodies to diffuse into the microwells 122a-d, where the markers 126 attach to the cells 124a-b retained in the microwells 122b-c. In some implementations, the cells 124a-b are washed (e.g., after flowing the solution containing fluorescent markers through the channel 120) by flushing the microfluidic device 101 with a buffer solution until residual antibodies diffuse out of the microwells 122a-d. In some implementations, the cells include monocytes from a sample of peripheral blood mononuclear cells (PBMCs). The monocytes can be labeled and captured without cell loss during fluidic operations.

The microwells 122a-d are high aspect ratio microwells that have a depth that is larger than their diameter. For instance, the depth of the microwells can be 3 to 10 times larger than the diameter. The depth can be between 50 and 150 micrometers, e.g., 100 micrometers, 110 micrometers, 90 micrometers, and so forth. The diameter can be between 10 and 100 micrometers, e.g., 10 μm, 20 μm, 30 μm, 40 μm 50 μm, 60 μm, 70 μm, 80 μm, 90 μm or 100 μm. While specific depths and diameters are listed, other depths and diameters are possible that result in vortices in the microwells during fluid medium recirculation.

The microfluidic device 101 is configured to culture the cells 124 in the microwells 122. For example, the cells can be cultured for an amount of time including hours, days, weeks, etc. The microwells 122 are configured to prevent tissue attachment to the walls of the microwells. For example, the microwells are fabricated from a plastic (PDMS) resistant to cell attachment. Once the cells are cultured for a sufficient amount of time, magnets are used to assist in removing the cells, as described in relation to FIG. 1C.

FIG. 1C shows steps of process 100 for removing the cells 124 (e.g., cells 124a-b) from the microwells 122a-d. At step 112, the microfluidic device 101 is flipped over (e.g., to its initial orientation, with the microwells 122a-d on the top of the device) and the magnet 130 is placed below the microfluidic device 101 and across from the openings of the microwells 122b-c. The cells 124a-b in the microwells are still labeled with the antibody-conjugated magnetic nanoparticles 128 and are pulled out of the microwells 122b-c by the magnetic force exerted by the magnet 130. At step 114, the cells 124a-b are flushed from the microfluidic device 101 by flushing the channel 120 with a solution (such as a buffer solution). The use of the magnet 130 enables specific groups of cells 124 to be removed from the microwells at any given time. In some implementations, the magnet 130 can be used to remove cells from any number of the microwells 122a-d for exporting them from the microfluidic device 101 at the same time.

Generally, a subset of cells can be selected in the microwells if the magnetic beads on the captured cells are removed from the cells. The cells are incubated with magnetic beads that are configured for selecting for a different surface marker. Removal of the captured magnetic beads can be performed in the microfluidic device.

Each cell generally expresses a number of surface markers that define the type and sub-type of the cell. For example, T-cells express CD3, while a subset of T-cells called cytotoxic T-cells additionally express CD8. CD3 cells can be isolated using CD3-labeled magnetic beads. The CD3 magnetic beads can then be removed from the captured cells. The captured cells can be labeled with CD8 magnetic beads. Last, the CD8 cells subset only, of the initial CD3 population of cells, can be removed using the CD8 magnetic beads.

In an example experiment, more than 5000 CD14+ cells were loaded into a microfluidic device such as the device 101. After performing steps 112 and 114, including the magnet unload and flush steps, no detectable CD14+ cells were detected in the microwells 122 of the microfluidic device 101, demonstrating that all cells captured in wells were able to be subsequently unloaded from wells and exported.

The microfluidic device 101 can be sealed to create a closed system. Specifically, cell isolation and maturation can be performed once the microfluidic device 101 is sealed from outside environmental factors. This enables cell isolation and maturation to be performed in non-sterile external environments, if the microfluidic device 101 remains sealed. The microwells 122 and the flow channel 120 can be patterned over a large area to handle a target number of cells 124. The geometry and positioning of the microwells 122 prevents flow through the channel 120 from displacing cells 124 in the microwells. As subsequently described, the wells are relatively tall and narrow in geometry, though different microwell sizes are possible. The microfluidic device 101 enables cell isolation performed with immunomagnetic isolation and inverted microwells 122, and surface marker characterization, culture, differentiation, and maturation are performed by reagent exchange in the channel 120. The cell culture is performed by perfusing media in flow channel 120. Cell harvest is performed by inverting wells to sediment out cells, as previously described.

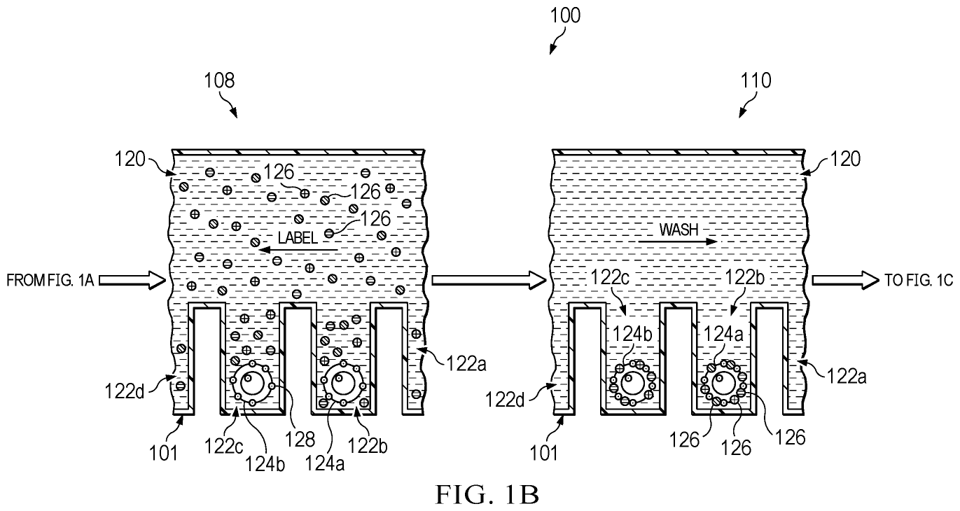

FIG. 2 is a flow diagram illustrating an example process 200 for cell isolation and reagent exchange in a microfluidic device, such as microfluidic device 101 of FIGS. 1A-1C. The process 200 shows a dendritic cell (DC) therapy manufacturing process and is a particular implementation of the process 100 of FIGS. 1A-1B. Cells, such as CD14+ monocyte cells, are introduced to the microwells of the microfluidic device in a fluid media (such as GM-CSF FL-t-3L). In process 200, at step 202, the media is configured to culture CD14+ monocyte cells 210, 212 in the wells of the microfluidic device. In an example, the monocyte cells 210, 212 are cultured for several days (e.g., 3 days).

A media pathogen is introduced to the microwells at step 204. The media pathogen includes cytokines IL-4, TNF-α, and/or PGE2+antigen. The antigen is taken up by the dendritic cells to promote an antigen-specific immune response in the patient. In step 206, a media including trypsin is circulated in the microfluidic device 101 including the retained cells 210, 212. The purpose of the trypsin is to assist with detachment of cells from the surface of the microfluidic device. After maturation of the cells 210, 212, CD83+, CD86+ cytokine secretion occurs. The surface markers plus cytokine secretion are signatures of mature, antigen-presenting dendritic cells that will promote an immune response in the patient. The cells 210, 212 are exported at step 208.

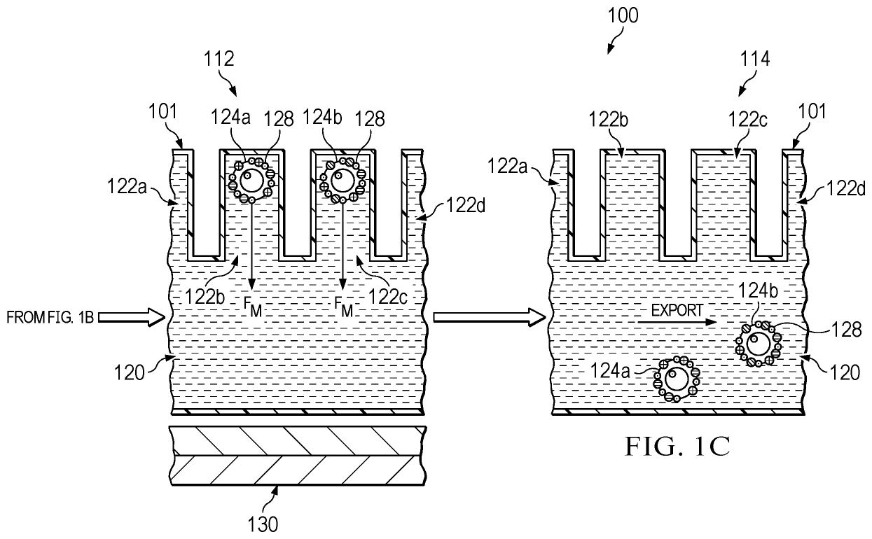

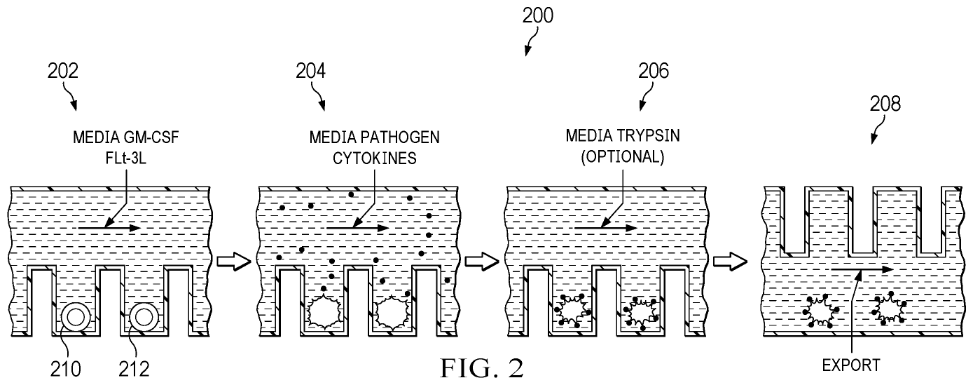

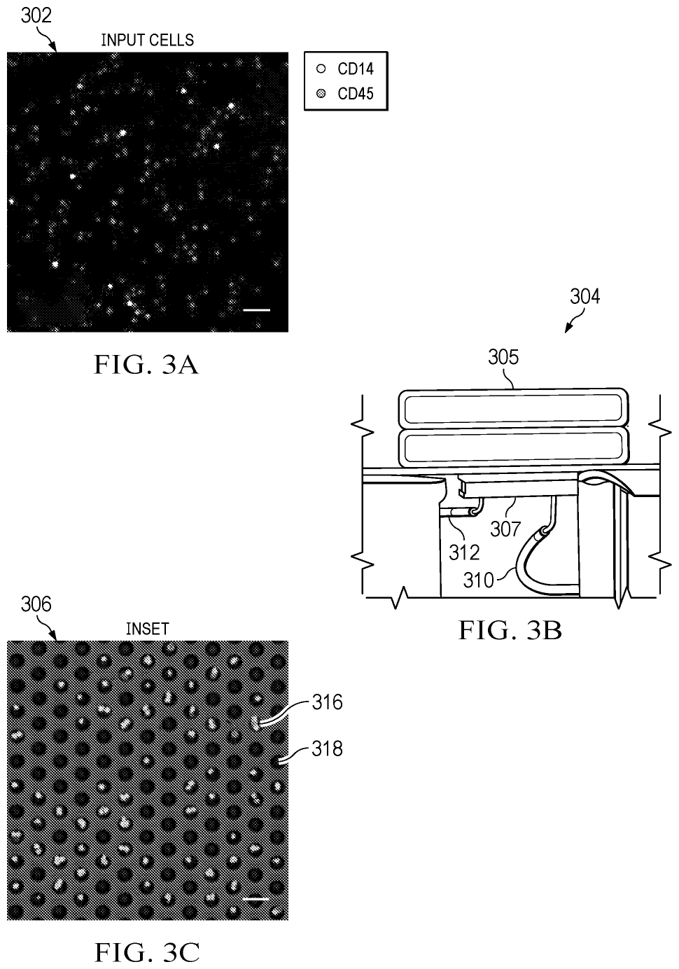

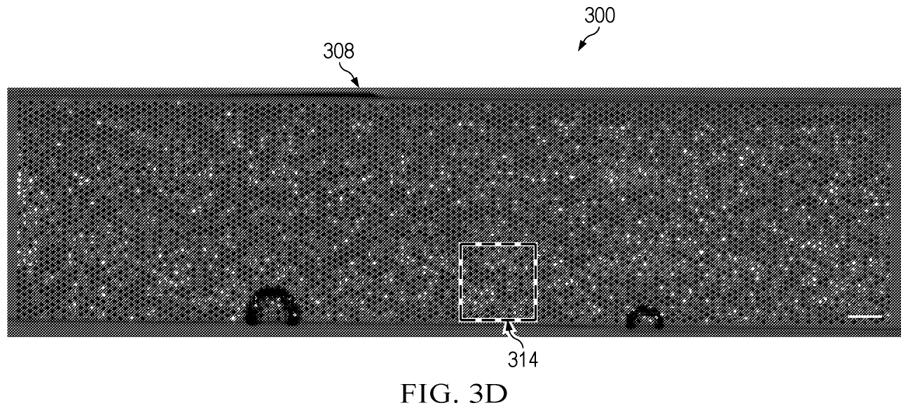

FIGS. 3A, 3B, 3C, and 3D each shows an image of cell retention in microwells of a microfluidic device 307, such as microfluidic device 101 of processes 100, 200 previously described in relation to FIGS. 1-2, respectively. In this example, high aspect-ratio microwells are used for immunomagnetic isolation of CD14+ monocytes from PBMCs with high purity over a wide range of input cell densities, shown in image 302. In this example, the microfluidic device 307 includes 5193 microwells each with a 30 micron (um) diameter and 100 micron depth. Here, an example microwell 318 is labeled. The wells 318 are patterned in a 2×8 millimeter (mm) area. A flow channel with a single inlet 310 and outlet 312 over the well area has a 200 micron depth. PMBCs are isolated from whole blood and labeled with CD14 magnetic nano-beads and fluorescent antibodies against CD14 (monocytes) and CD45 (differentiated hematopoietic cells) surface markers. Density, viability, and CD14 abundance are measured in the input cell sample of image 302. A cell sample was pulled into a microfluidic device 307 in a first, wells-up configuration. After pulling cells into the microfluidic device 307, two stacked 32 pound (lb.) pull-force neodymium magnets 305 are placed on top of the microfluidic device 307, as shown in image 304. The magnets 305 are placed on the microfluidic device 307 for about 10 minutes to pull bead-labeled cells into the microwells 318. After about 10 minutes, the cells remaining in the channel are flushed with 1 milliliter (mL) of a buffer solution at 100 microliters per second (μL/s). The magnets 305 are removed, and the microfluidic device 307 is flipped to retain captured cells by gravity (a wells-down configuration).

After cell capture, a composite brightfield/fluorescent image 306 is captured of the area of microwells 318 of the microfluidic device 307. Cells (including cells labeled 316) are counted in each microwell 318, such as using a semi-automated program, to determine a total number of CD45+ cells and CD14+/CD45+ cells. A larger image 308 shows the microfluidic device 300 with the area of image 306 shown in a box 314.

In the example of FIG. 3, the cells can include PBMCs isolated from whole blood and labeled with Miltenyi CD14 microbeads and antibodies against CD14 (monocytes) and CD45 (differentiated hematopoietic cells) surface markers. Here, the scale bar is 50 microns for images 302 and 306. For image 308, the scale bar is 250 μm. Image 308 includes a composite brightfield/CD14/CD45 microscopic image of a 5193 microwell microfluidic device after monocyte capture. In an example, from an input PBMC sample containing 19% CD14+/CD45+ cells, 3123 cells are captured with 98% CD14+/CD45+ purity.

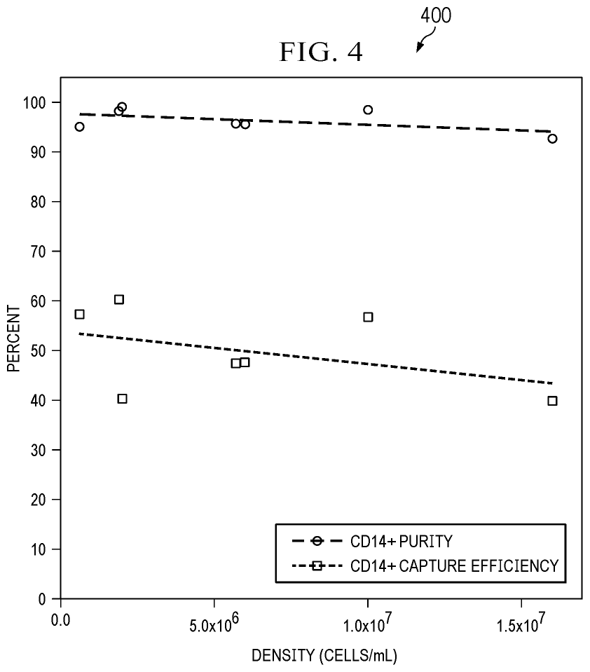

Results from immunomagnetic separation experiments performed over a range of input cell density are summarized in Table 1. Captured CD14 cell purity as well as effective capture efficiency are plotted in graph 400 of FIG. 4. Seven experiments were performed with a range of input cell densities from 6.2×10⁵ to 1.6×10⁷ cells/mL, and an average purity of 962% is observed with average capture efficiency of 508%. Purity is defined as number of captured CD14+/CD45+ cells divided by number of captured CD45+ cells. Capture efficiency is defined as number of captured CD14+/CD45+ cells divided by the estimated number of CD14+ cells available in the microfluidic device during a separation (cell density×viability×CD14% in PBMC input×device volume). Graph 400 shows that cells labeled with an off the shelf magnetic nanoparticle kit are separated with relatively high purity (e.g., more than 90%) and consistent capture efficiency (e.g., between 40% and 60%) across a range of input densities.

TABLE 1
Example Immunomagnetic Separation Results
Input Characteristics

| | | | | | | | |
|---|---|---|---|---|---|---|---|
| Density (cells/mL) | 6.2E+5 | 1.9E+6 | 2.0E+6 | 5.7E+6 | 6.0E+6 | 1.0E+7 | 1.6E+7 |
| Viability (%) | 85 | 88 | 77 | 92 | 77 | 95 | 96 |
| CD14+ Purity (%) | 13 | 13 | 31 | 13 | 31 | 19 | 35 |
| CD14+ cells on | 201 | 637 | 1432 | 1998 | 4297 | 5415 | 16215 |
| microfluidic device | | | | | | | |
| (est.) | | | | | | | |

Captured Cell Characteristics

| | | | | | | | |
|---|---|---|---|---|---|---|---|
| Microwells with cells | 91 | 307 | 483 | 692 | 1434 | 2074 | 3382 |
| CD45+ cells | 121 | 391 | 584 | 992 | 2142 | 3123 | 6981 |
| CD14+/CD45+ | 115 | 384 | 578 | 948 | 2045 | 3073 | 6465 |
| CD14+ Purity (%) | 95 | 98 | 99 | 96 | 95 | 98 | 93 |
| Capture Efficiency (%) | 57 | 60 | 40 | 47 | 48 | 57 | 40 |
| (est.) | | | | | | | |

FIG. 4 is a graph 400 illustrating cell retention in microwells of a microfluidic device. CD14+ purity and capture efficiency are shown as a function of input cell density. Over seven experiments with input cell densities ranging from 6.2×10⁵ to 1.6×10⁷ cells/mL, an average purity of 962% is observed with average capture efficiency of 508%. A linear fit of the two parameters shows that purity and capture efficiency do not depend strongly on input cell density.

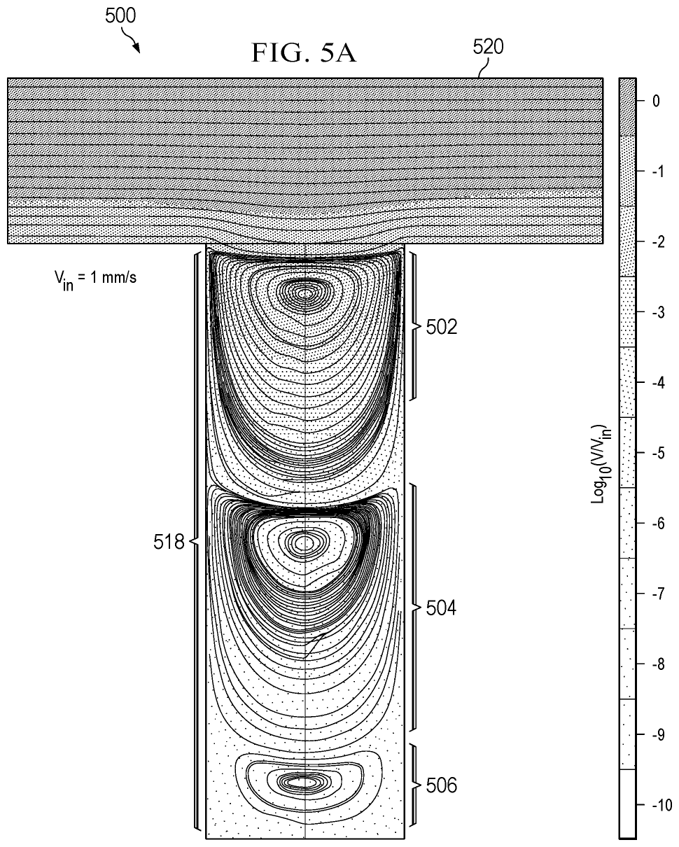

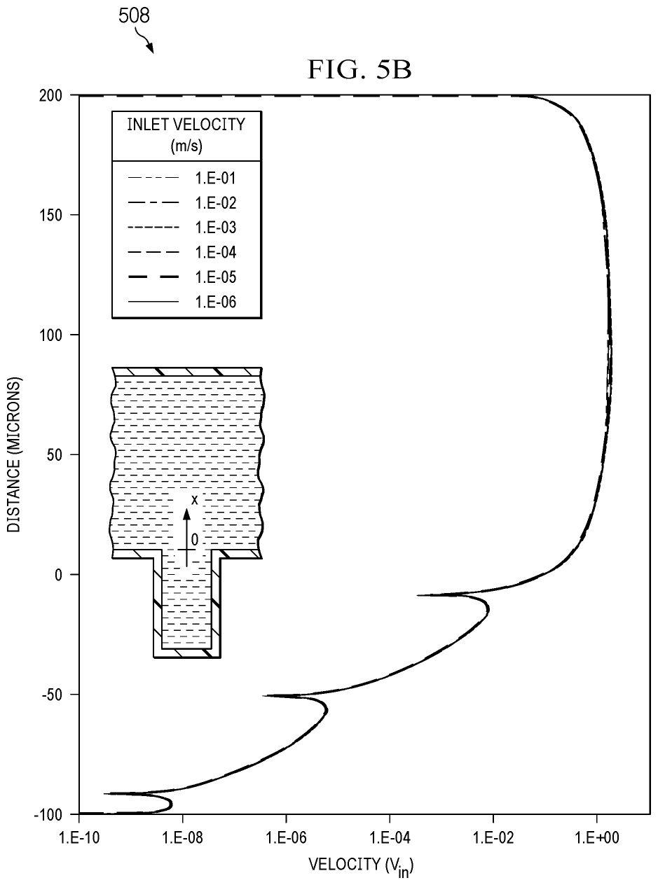

FIG. 5A includes a model 500 showing a simulation of fluid velocity in a microwell of the microfluidic device. For the microfluidic devices described herein, the microwells allow multi-reagent operations on to be performed on captured cells without cell loss. Cells enter the wells by non-fluidic forces and are retained in the wells after capture due to flow recirculation at the entrance of the well and rapidly decreasing fluid velocity deeper into the well.

For simulation in model 500, the fluid flow is simulated in a geometry composed of a single cylindrical well 518 with a 30 micron diameter and a 100 micron depth. The well 518 is connected to a 200 micron deep channel 520 with average inlet velocity swept from 1 um/s to 10 centimeters per second (cm/s) in ten intervals. Image 500 shows a normalized velocity magnitude and velocity streamlines for an example inlet velocity of 1 millimeter per second (mm/s). Streamlines show that flow from the channel does not enter the well 518 as the fluid moves across an opening of the well 518 at the channel 520. Rather, a series of recirculating vortices 502, 504, 506 are generated starting at a top of the well 518 near vortex 502. A streamline pattern observed at the example velocity spans a practical operating range of microfluidic device wells from fast flushes to perfusion for culture. Cells or objects driven by flow in the main channel follow streamlines unless acted on by an external force. A cell enters or exits the well 518 through non-fluidic forces (e.g., magnetic, gravitational, thermal, etc.) rather than fluidic forces form the fluid flow of the channel.

Normalized velocity profiles from the simulation indicates that velocity decreases exponentially with increasing depth into the well 518. Velocity profiles through the center of the well 518 are normalized to the inlet velocity and are shown in graph 508. Graph 608 shows inlet velocities ranging from 1 micron/s to 10 cm/s. The profiles are nearly identical for all inlet velocities and show an exponential decrease in velocity with increasing depth in the well 518. For the well geometry of well 518, a velocity of fluid in a bottom of the well 518 (near vortex 506) is reduced by a factor greater than 108 from a velocity of fluid flow in the channel 520. Cells or beads in the bottom of wells such as well 518 experience essentially no flow even when fluid is moving quickly (10 cm/s) in the channel 520. A static fluid environment enables a low-shear environment for cell culture and for ensuring that reagent exchange is performed without risk of cell loss.

In image 500, the velocity streamlines are shown as black lines. The shaded map shows a velocity magnitude normalized to an inlet velocity in a log scale. The streamlines show that flow from the channel 522 does not enter the well 518. Instead, there are a series of three counter-rotating vortices 502, 504, 506. As a result, cells or other objects cannot enter the well 518 due to fluid flow alone.

Flow behavior within wells is independent of flow rate. Simulations of flow within a 30 micron diameter well with 100 micron depth at flow rates from 10 centimeters per second (cm/s) to 1 μm/s show nearly identical streamline patterns as well as velocity magnitude normalized to inlet velocity.

FIG. 5B shows a graph that 508 shows velocity profiles through a center of the well 518. The velocity profiles are normalized to the inlet velocity. The velocity profiles show an exponential decrease in fluid velocity as depth into the well increases. The normalized velocity profiles are nearly identical for all inlet velocities ranging from 1 micron/s to 10 cm/s and the velocity at the bottom of the well is decreased by a factor greater than 10⁸.

Cell retention during fluidic operations are experimentally verified by exposing beads in wells to a sequence of flushes at representative flow rates and monitoring for bead loss. 207 magnetic beads with a diameter of 4 microns were captured into wells using the same process described for capturing cells. The microfluidic device is flushed with 1 mL of buffer at 100 microliters per second (μL/s), imaged, and beads are counted. This is repeated for 10 μL/s, 1.0 μL/s and 0.1 μL/s in sequence. During this process, which exposed beads to flow rates varying over three orders of magnitude and greater than three hours of flushing, 207 beads were observed after every step, and no beads were lost. The process and results are summarized in Table 2. Therefore, beads or cells captured in wells are not lost to flow in the channel during reagent exchange.

TABLE 2
Bead retention experimental process and results.

| | Flow | Mean Channel | | | |
|---|---|---|---|---|---|
| | Rate | Velocity | | Beads | Beads |
| Step | (μL/s) | (mm/s) | Time | Counted | Lost |
| Capture | — | — | — | 207 | — |
| 1 mL flush | 100 | 250 | 10 s | 207 | 0 |
| 1 mL flush | 10 | 25 | 1 m 40 s | 207 | 0 |
| 1 mL flush | 1.0 | 2.5 | 16 m 40 s | 207 | 0 |
| 1 mL flush | 0.1 | 0.25 | 2 h 46 m | 207 | 0 |

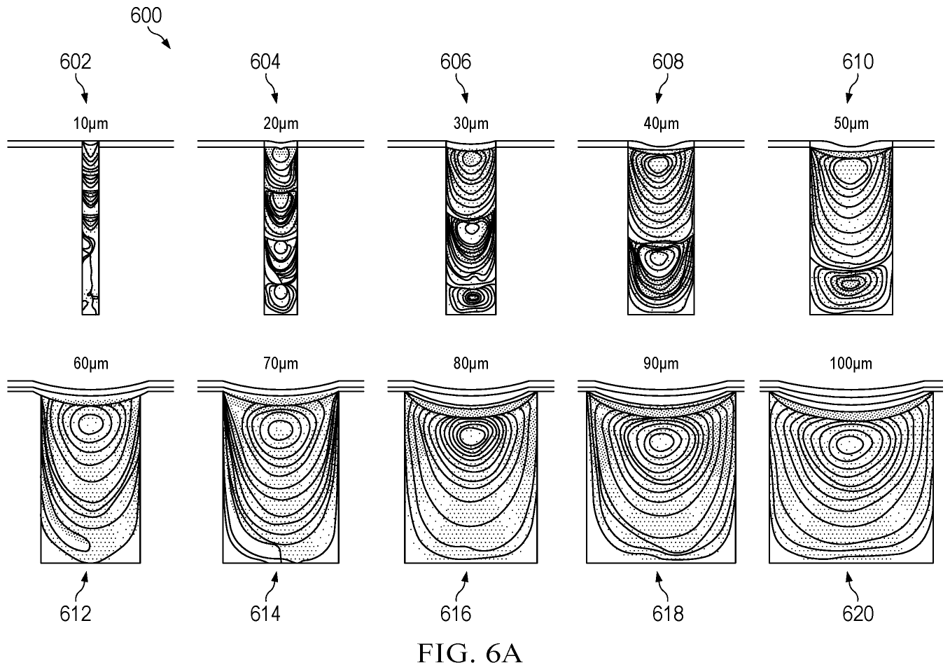

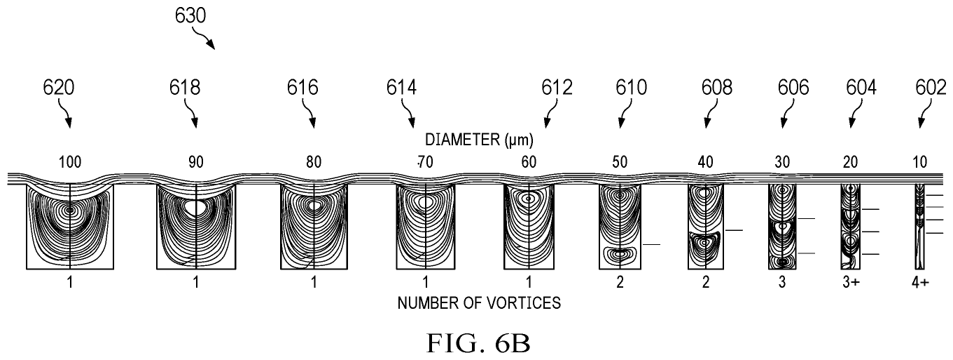

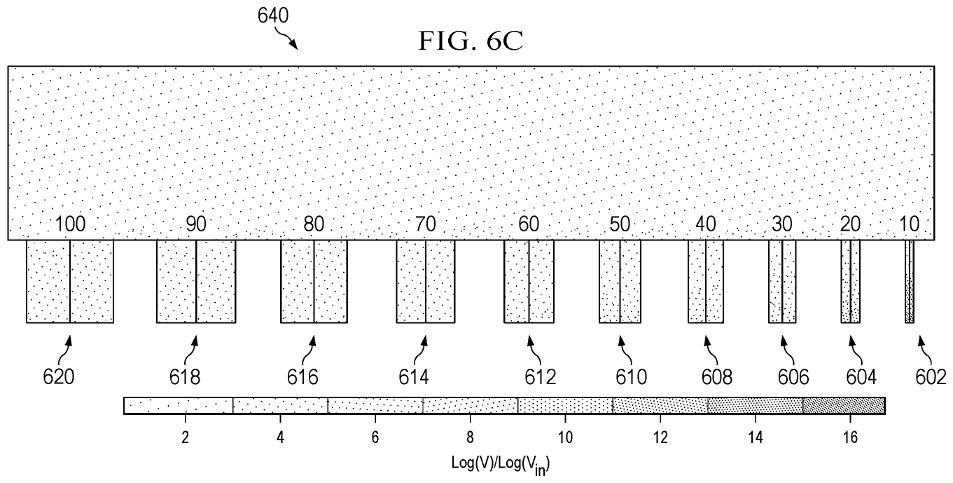

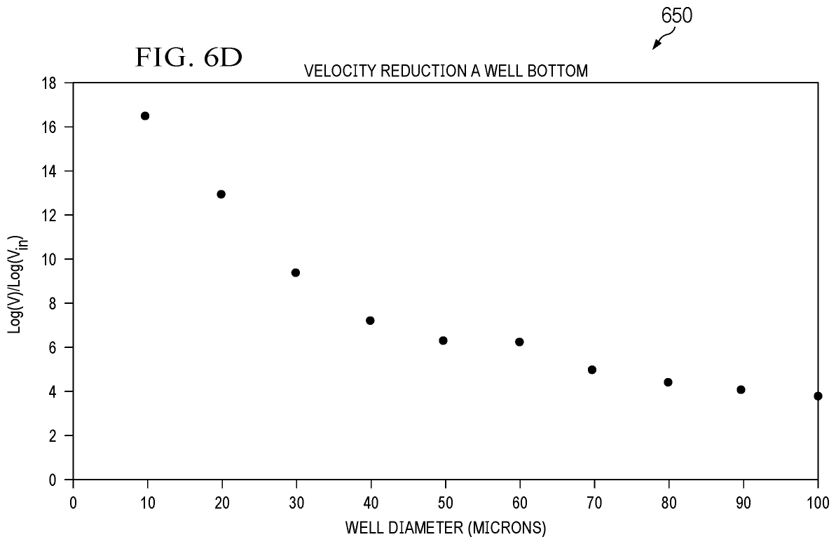

FIG. 6A shows simulations of fluid velocity for microwells 600 having a variety of diameters. In FIG. 6A, the microfluidic devices each have a channel layer configured for flow of a fluid medium and a plurality of microwells in fluid communication with the channel layer, the plurality of microwells each including a well depth and a well diameter that prevents fluid flow of the fluid medium into the plurality of microwells during circulation of the fluid medium in the channel layer. Generally, the well depth and the well diameter of each of the plurality of microwells are each a function of a flow rate of the fluid medium in the channel layer. For a given microwell, a given well depth and a given well diameter of the given microwell together cause fluid medium present in the given microwell to form one or more vortices during circulation of the fluid medium in the fluid channel. One or more vortices prevent the fluid medium from flowing into the given microwell from the channel layer. For each of the wells 602, 604, 606, 608, 610, 612, 614, 616, 618, and 620 shown in FIG. 6A, a well depth is about 100 micrometers. The flow rate of the fluid medium is between 1 micrometer per second and 10 centimeters per second. The well diameter is 10 micrometers for well 602. The well diameter is 20 micrometers for well 604. The well diameter is 30 micrometers for well 606. The well diameter is 40 micrometers for well 608. The well diameter is 50 micrometers for well 610. The well diameter is 60 micrometers for well 612. The well diameter is 70 micrometers for well 614. The well diameter is 80 micrometers for well 616. The well diameter is 90 micrometers for well 618. The well diameter is 100 micrometers for well 620.

Generally, for wells 602, 604, and 606, the respective well diameters of 10-30 micrometers cause three or more vortices in the microwells during the circulation of the fluid medium. Generally, for wells 608 and 610, the respective well diameters of 40-50 micrometers cause two vortices in the microwells during the circulation of the fluid medium. Generally, for wells 612-620, the respective well diameters of 60-100 micrometers cause one vortex in the microwell during the circulation of the fluid medium.

FIG. 6B shows simulations 630 of fluid velocity for microwells having a variety of diameters. The microwells 602-620 are similar to the microwells of FIG. 6A. As shown in FIG. 6B, microwell 602, having a well diameter of 10 micrometers, has four vortices form in the microwell during fluid circulation of the fluid medium.

FIG. 6C shows simulations 640 of fluid velocities during a cell sweep process for microwells having a variety of diameters. The shading shows a fluid velocity ratio of the fluid in the microwells and channel with respect to a fluid inlet velocity. The graph 640 of FIG. 6D shows the results of the fluid velocity reduction during a cell sweep process for microwells of FIG. 6C. Generally, three or more vortices are preferred to prevent fluid flow from the channel into the microwells.

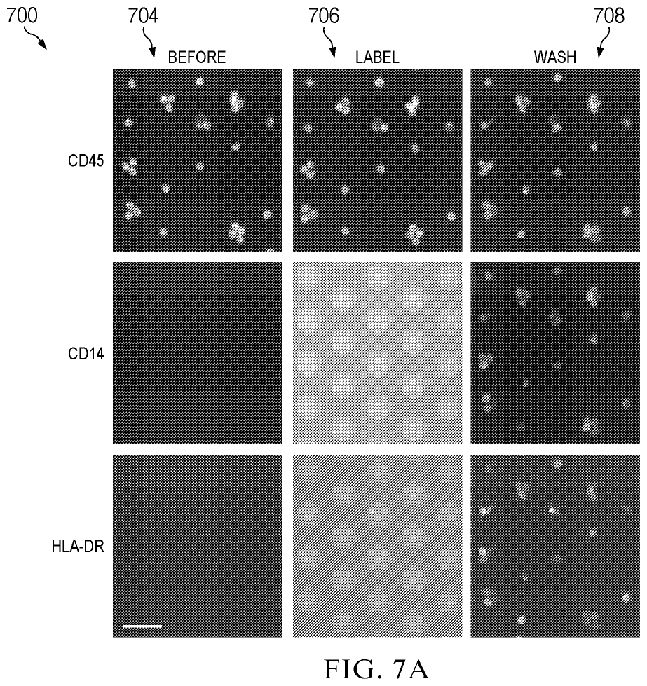

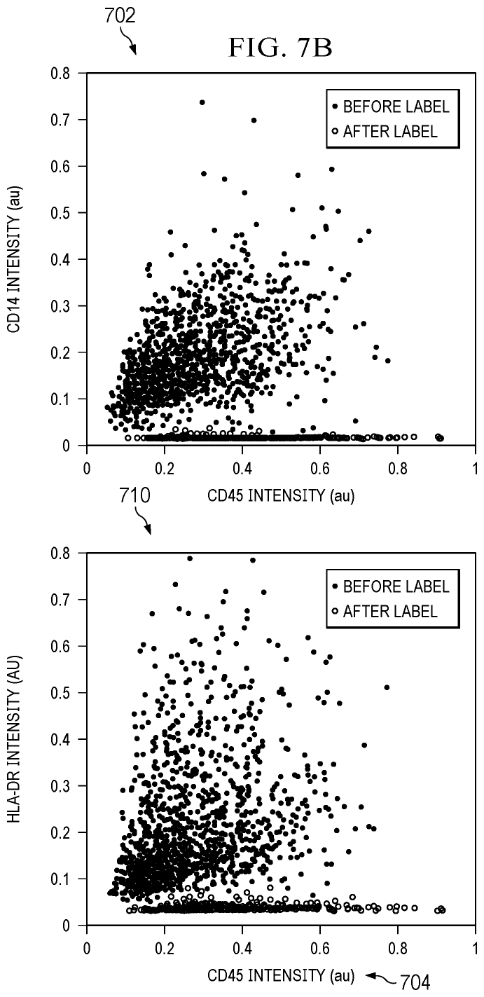

FIG. 7A shows images 700 of a cell marking process using the microwells of FIGS. 1-6C. Experimental operations on cells, such as labeling cells with fluorescent antibodies, can be performed on the microfluidic device (e.g., on-chip) because of the microfluidic device's ability to retain cells a composition a fluid medium in the microfluidic device is changed. In images 700, a CD14 isolation process is shown. Cells are only labeled for CD45 and labeling them on-chip with antibodies against CD14 and HLA-DR. Generally, low or negative expression of HLA-DR has been associated with an immunosuppressive state that can hinder dendritic cell maturation. The images 700 show a cropped region of a microfluidic device including the microwells described herein. Cells are in each fluorescent channel (CD45/CD14/HLA-DR) before labeling 704, during labeling 706, and after washing 708. Cells that were initially unlabeled at step 704 for CD14 and HLA are subsequently labeled in the wells. A significant fluorescent background is shown at the labeling step 706. The fluorescent background is removed after the wash at step 708. This allows fluorescence intensity of individual cells to be characterized. The scale bar is 50 μm.

FIG. 7B shows graphs 702, 710, which each includes a scattergram (CD45 vs CD14 and CD45 vs HLA-DR, respectively) of fluorescence intensity for 3123 CD45+ cells identified after immunomagnetic isolation before and after labeling, with clear separation between unlabeled and labeled populations observed.

### Example Experiment

### Microfluidic Device Fabrication

Microfluidic devices were designed using computer-aided design (CAD) software and were fabricated in PDMS using photolithography and soft lithography. The microwell devices include a microwell layer with 5193 cylindrical microwells and a channel layer for fluid transport over the microwells. Microwells had 30 micron diameters and depth of 100 microns and the channel layer is 200 microns thick. Replica molded PDMS layers were aligned and bonded to each other and a glass slide by oxygen plasma (YES-G1000, Yield Engineering Systems). Fluidic connections were made by through-holes punched in the channel layer.

### Cell Preparation

Cell samples were obtained from blood donated by volunteers at the Division of Transfusion Medicine, Mayo Clinic, Rochester, Minnesota, in accord with current regulations by the AABB and US FDA. Following an apheresis procedure, a leukoreduction system chamber from the apheresis apparatus (Trima Accel, Gambro BCT, Lakewood, CO) was collected, and PBMCs were isolated using density gradient separation in Histopaque (Sigma Aldrich) following a previously established protocol. Cell density and viability were enumerated using a Countess Cell Counter (Thermo Fisher) and trypan blue exclusion. Cells were labeled with CD14 MicroBeads (Miltenyi) following the manufacturer's protocol and stained with antibodies (Biolegend) against CD45 and CD14 surface markers before being centrifuged, washed and re-suspended to a desired cell density. In the on-device labeling experiment, cells were only labeled with CD45 antibodies before on-device capture.

### Device Operation

Devices were wet with a solution of 1% (w/v) Pluronic F-127 in PBS with fluid flow driven by a hand-operated syringe or syringe pump (KD Scientific). On-chip magnetic isolation was performed by pulling labeled cell suspensions into the flow channel, stopping flow, and placing a magnet on top of an inverted microfluidic device. Two 32 pound (lb.) pull force neodymium magnets (K&J Magnetics, USA) were stacked and placed on top of the chip for 10 minutes as shown in FIG. 1A, then the flow channel was flushed with greater than 1 mL of buffer at approximately 100 μL/s. The magnets were removed, and the chip was flipped to a wells-down configuration for imaging and other fluidic operations.

### Microscopy & Image Analysis

Imaging was performed on an EVOS Cell Imaging system. Stitched, composite brightfield and fluorescent images were captured over the entire well region for each device. Individual channels (brightfield and fluorescent) were split for semi-automated analysis in MATLAB. Individual wells were identified using the circular Hough transform and used to mask fluorescent images. After adjusting for brightness/contrast, cell quantification was performed on the masked fluorescent images and detected cells were assigned to a specific well. Any well that contained a fluorescent cell in any channel was then manually inspected to confirm the count. The resulting data included total number of cells detected for each fluorescent channel and the number of cells per well.

### On-Chip Labeling

Antibodies were diluted 10:1 in buffer, pulled onto the chip, incubated for 10 minutes at room temperature, and subsequently flushed from the chip. Washing was performed by flushing the chip with buffer for 10 s at 100 μL/s and then 30 min at 1 μL/s. The initial flush is to remove the label from the main channel while the secondary flush allows antibodies within the well time to diffuse out. Full chip images were captured in fluorescent channels corresponding to each antibody before staining, during labeling, and after washing. Exposure times were constant in CD45 channel (500 milliseconds (ms)) across all steps while exposure times were reduced during the labeling step in the CD14 (500 ms to 20 ms) and HLA-DR (300 ms to 100 ms) channels before being returned to initial values in the post-wash image.

### Example Experiment: Dendritic Cell (DC) Culture

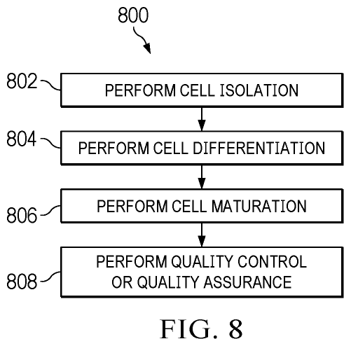

FIG. 8 shows an example process 800 for DC culture on a microfluidic device, such as the microfluidic device 101 described herein. Inputs to the process 800 include PBMCs, and outputs include the DCs. The microfluidic device 101 is configured for integration of monocyte isolation, differentiation, and maturation on the enclosed microfluidic device. In the DC culturing process, the user performs (802) cell isolation in the microwells. This includes positive immunomagnetic selection. Positive selection include selecting a desired cell population with an antibody specific to a cell surface marker. Positive immunomagnetic selection includes positive cell selection using antibodies-coated paramagnetic beads that bind to antigens present on the surface of cells. The cells are captured in the microwells when the magnetic force is applied, facilitating the concentration of these bead-attached cells, as described previously. In an example, cell isolation includes CD14+ monocyte isolation. The user performs (804) cell differentiation in the microfluidic device 101. The user can allow the cells to culture over time, such as 3 days culture and GM-CSF, IL-4.

During the cell differentiation process, media is perfused through the channel above the wells. The media perfusion allows fresh media to diffuse into wells while metabolic waste can diffuse from the microwells into the channel and be removed from the microfluidic device 101. Removal of metabolic waste maintains cell culture conditions and promotes differentiation.

The process 800 includes performing (806) cell maturation. For example, 2 days of cell culture and GM-CSF, IL-4, TNF-α, PGE2, and antigens are added. The user performs (808) quality control or quality assurance for the DCs. The percentage of viable cells is additionally a criteria used to assess the quality of the cells. Quality control or analysis includes CD83+, CD86+, and cytokine secretion.

Dendritic cell culture can be performed with the microfluidic device (e.g., microfluidic device 101), as described in the following experiment. CD14 isolation was performed on the microfluidic device 101. The microfluidic device 101 was coated with fibronectin. The fibronectin coating promotes cell adhesion that is needed to differentiate from monocytes to immature dendritic cells. The microfluidic device is then set to perfusion culture with differentiation media for 3 days, followed by culture for culture with maturation media for 2 days. Cells in the microwells of the microfluidic device 101 were characterized on-chip with fluorescent antibodies for CD45, CD14, and CD83 surface markers on Day 0 and Day 5. The cells were expected to express high CD14 (monocyte marker) and low CD83 (mature dendritic cell marker) on day 0. The cells were expected to express low CD14 (monocyte marker) and high CD83 (mature dendritic cell marker) on day 5. In an example, fewer than 4000 cells were loaded with ˜95% CD14 purity, and fewer than 1000 CD83+ cells were observed on day 5.

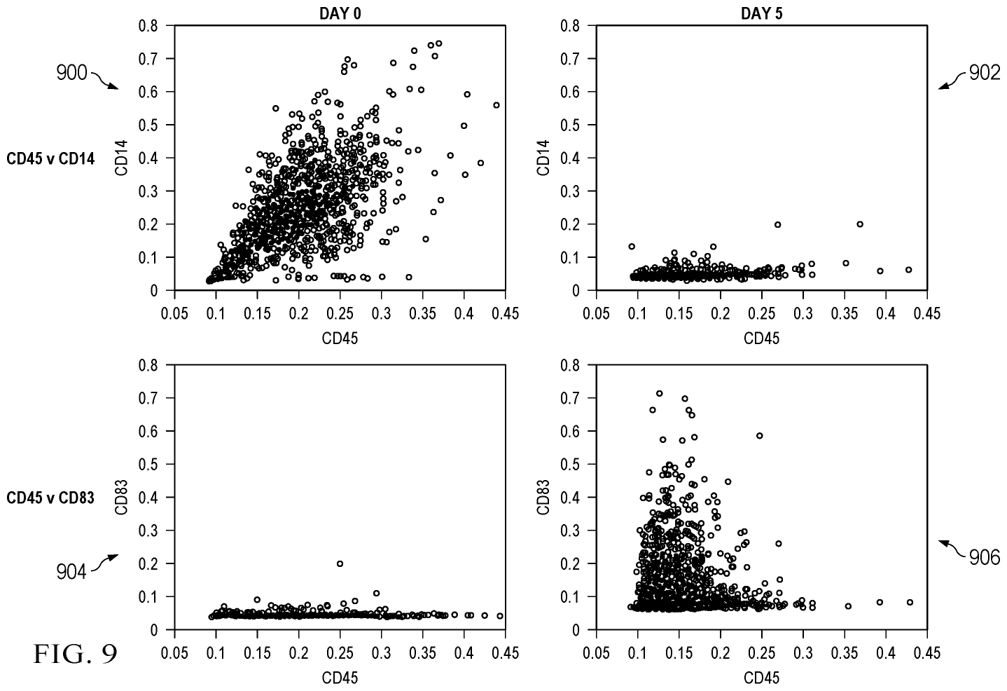

FIG. 9 includes scatterplots 900, 902, 904, and 906 showing results of the measurements taken for this example cell culture. Scatterplot 900 shows normalized expression values for CD45 and CD14 at day 0. Scatterplot 902 shows normalized expression values for CD45 and CD14 at day 5. Scatterplot 904 shows normalized expression values for CD45 and CD83 at day 0. Scatterplot 906 shows normalized expression values for CD45 and CD83 at day 5.

At day 0, there was a relatively high CD14 expression, and relatively low CD83 expression (or no CD83 expression). At day 5, there was low CD14 expression (or no CD14 expression. There were 1028 cells with CD83 expression greater than 0.1 out of 3629 CD45 cells detected (28%).

Cell separation can be performed using various surface markers. For example, high purity separation of CD14 monocytes can be performed using Miltenyi CD14 microbeads. Immunomagnetic isolation of cells with other surface markers can be performed for one or more of the following examples. T cells can be used for CD3, CD4, and CD8 cell types. NK cells can be used for CD56 cell types. Stem cells can be used for CD34 cell types.

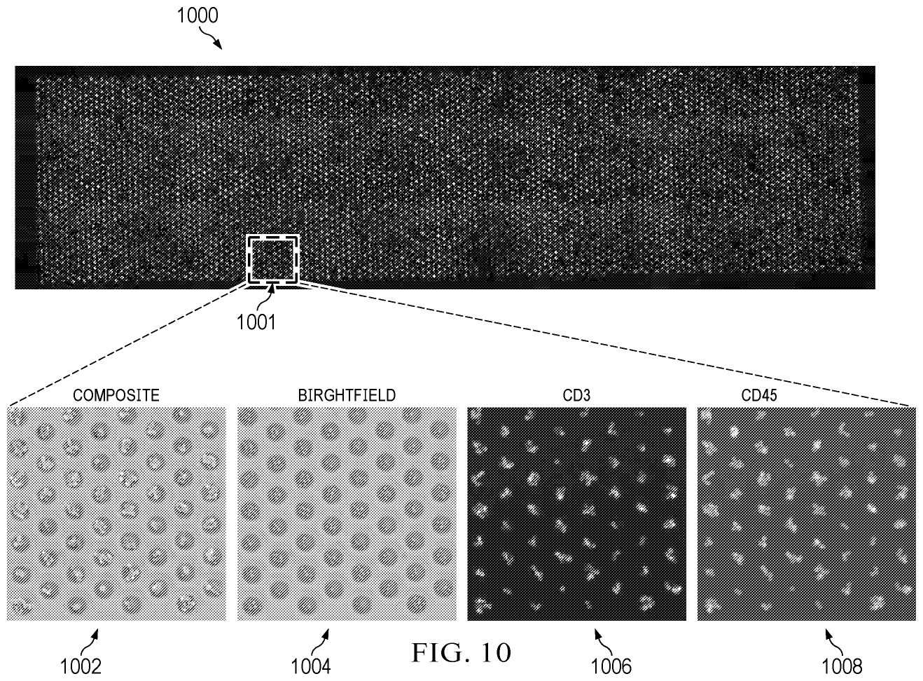

FIG. 10 shows an example of DC cell isolation on a microfluidic device (e.g., microfluidic device 101, previously described). The microfluidic device 101 has microwells 1000 including cells. Sample wells 1001 are shown in images 1002, 1004, 1006, and 1008. Image 1002 shows the sample wells 1001 with a composite of all the cell types included. Image 1004 shows a brightfield image of the sample wells 1001. Image 1006 shows CD3 cells in the sample wells 901. Image 908 shows CD45 cells in the sample wells 1001. For generating these images, the number of cells input into the array of microwells 1000 was 1.7×10⁷ cells/mL. There was 94% cell viability. 65% of the cells were CD3. On the microfluidic device 101, 23047 cells were captured, and 97% were CD3. There was a 73% capture efficiency. These results are estimated by extrapolating from cell count in 500 of 5193 wells.

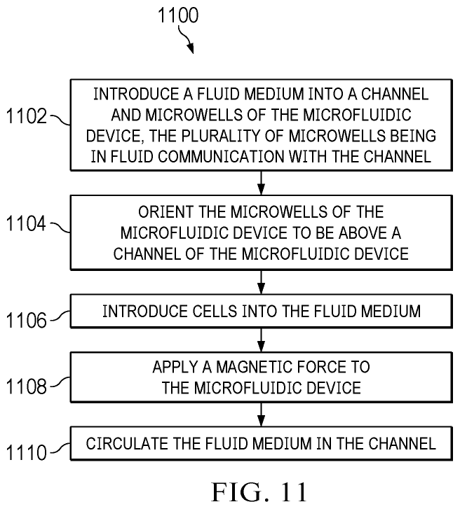

FIG. 11 shows a flow diagram showing an example process 1100 for culturing cells in a microfluidic device, such as the microfluidic device 101 described herein. The process 1100 can include labeling cells with a microfluidic device. The process 1100 includes introducing (1102) a fluid medium into a channel and microwells of the microfluidic device, the plurality of microwells being in fluid communication with the channel. The process 1100 includes orienting (1104) the microwells of the microfluidic device to be above a channel of the microfluidic device. The process 1100 includes introducing (1106) cells into the fluid medium, wherein a first portion of the cells are labeled with magnetic particles, and wherein a second portion of the cells are not labeled with the magnetic particles. The process 1100 includes applying (1108) a magnetic force to the microfluidic device to pull the first portion of the cells into the microwells, wherein the second portion of the cells remain in the channel. The process 1100 includes circulating (1110) the fluid medium in the channel to remove the second portion of the cells, wherein a geometry of the microwells prevents flow of the fluid medium from the channel into the microwells.

In some implementations, the geometry of the microwells that prevents flow of the fluid medium from the channel into the microwells is determined based on an expected flow rate of the fluid medium. In some implementations, the flow rate is between 1 micrometer per second and 10 centimeters per second. In some implementations, the geometry of the microwells that prevents flow of the fluid medium from the channel into the microwells comprises a microwell width and a microwell depth. In some implementations, the microwell width is 30 micrometers, wherein the well depth is 100 micrometers, and wherein the channel depth is 200 micrometers. In some implementations, the geometry of the microwells that prevents flow of the fluid medium from the channel into the microwells causes one or more vortices to form from fluid medium in at least one of the microwells during circulation of the fluid medium, the one or more vortices preventing fluid medium from flowing from the channel into the at least one microwell.

In some implementations, the process 1100 includes performing cell labeling of the first portion of the cells by circulating a second fluid medium including fluorophore conjugated antibodies into the channel and allowing the fluorophore conjugated antibodies to diffuse into the microwells.

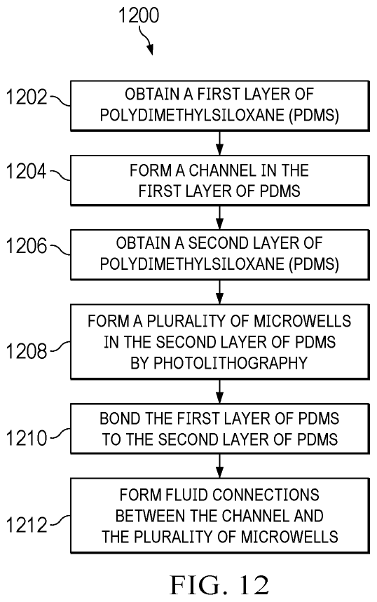

FIG. 12 shows a process 1200 for forming a microfluidic device, such as the microfluidic device 101 described herein. The process 1202 includes obtaining (1202) a first layer of polydimethylsiloxane (PDMS). The process 1200 includes forming (1204) a channel in the first layer of PDMS. The process 1200 includes obtaining (1206) a second layer of polydimethylsiloxane (PDMS). The process 1200 includes forming (1208) a plurality of microwells in the second layer of PDMS by photolithography, the plurality of microwells having a well diameter and a well depth configured to prevent fluid flow of the fluid medium into the plurality of microwells during circulation of the fluid medium in the channel layer. The process 1200 includes bonding (1210) the first layer of PDMS to the second layer of PDMS. The process 1200 includes forming (1212) fluid connections between the channel and the plurality of microwells. In some implementations, the fluid connections are formed by holes punched in the first layer.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

In the foregoing description, embodiments of the processes and systems described herein are described with reference to numerous specific details that may vary from implementation to implementation. The description and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction. Any definitions expressly set forth herein for terms contained in such claims shall govern the meaning of such terms as used in the claims. In addition, when we use the term “further comprising” or “further including” in the foregoing description or following claims, what follows this phrase can be an additional step or entity, or a sub-step/sub-entity of a previously-recited step or entity.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

A number of embodiments of these systems and methods have been described. Nevertheless, it will be understood that various modifications may be made without departing from the spirit and scope of this disclosure.

1. A microfluidic device, comprising:
a channel layer configured for flow of a fluid medium; and
a plurality of microwells in fluid communication with the channel layer, the plurality of microwells each comprising a well depth and a well diameter that prevents fluid flow of the fluid medium into the plurality of microwells during circulation of the fluid medium in the channel layer.

2. The microfluidic device of claim 1, wherein the well depth and the well diameter of each of the plurality of microwells are each determined based on an expected flow rate of the fluid medium in the channel layer.

3. The microfluidic device of claim 1, wherein, for a given microwell, the well depth and the well diameter of the given microwell together cause fluid medium present in the given microwell to form one or more vortices during circulation of the fluid medium in the fluid channel, wherein the one or more vortices prevent the fluid medium from flowing into the given microwell from the channel layer.

4. The microfluidic device of claim 1, wherein the well depth is 100 micrometers, wherein a flow rate of the fluid medium is between 1 micrometer per second and 10 centimeters per second, and wherein the well diameter is one of:
10-30 micrometers causing three or more vortices in the given microwell during the circulation of the fluid medium;
40-50 micrometers causing two vortices in the given microwell during the circulation of the fluid medium; or
60-100 micrometers causing one vortex in the given microwell during the circulation of the fluid medium.

5. The microfluidic device of claim 1, further comprising a magnet configured to pull cells labeled with magnetic nanoparticles into the plurality of microwells during a cell separation process.

6. The microfluidic device of claim 1, wherein the channel layer is 200 micrometers thick.

7. The microfluidic device of claim 1, wherein the well depth is approximately 100 micrometers.

8. The microfluidic device of claim 1, wherein the well diameter is between 10-100 micrometers.

9. The microfluidic device of claim 1, wherein the channel layer and microwells are formed from polydimethylsiloxane (PDMS).

10. A method of labeling cells with a microfluidic device, the method comprising:
introducing a fluid medium into a channel and microwells of the microfluidic device, the microwells being in fluid communication with the channel;
orienting the microwells of the microfluidic device to be above a channel of the microfluidic device;
introducing cells into the fluid medium, wherein a first portion of the cells are labeled with magnetic particles, and wherein a second portion of the cells are not labeled with the magnetic particles;
applying a magnetic force to the microfluidic device to pull the first portion of the cells into the microwells, wherein the second portion of the cells remain in the channel; and
circulating the fluid medium in the channel to remove the second portion of the cells, wherein a geometry of the microwells prevents flow of the fluid medium from the channel into the microwells.

11. The method of claim 10, wherein the geometry of the microwells that prevents flow of the fluid medium from the channel into the microwells is determined based on an expected flow rate of the fluid medium.

12. The method of claim 10, wherein the flow rate is between 1 micrometer per second and 10 centimeters per second.

13. The method of claim 10, wherein the geometry of the microwells that prevents flow of the fluid medium from the channel into the microwells comprises a microwell width and a microwell depth.

14. The method of claim 10, wherein the microwell width is 30 micrometers, wherein the microwell depth is 100 micrometers, and wherein a channel depth is 200 micrometers.

15. The method of claim 10, wherein the geometry of the microwells that prevents flow of the fluid medium from the channel into the microwells causes one or more vortices to form from fluid medium in at least one of the microwells during circulation of the fluid medium, the one or more vortices preventing fluid medium from flowing from the channel into the at least one microwell.

16. The method of claim 10, further comprising performing cell labeling of the first portion of the cells by circulating a second fluid medium including fluorophore conjugated antibodies into the channel and allowing the fluorophore conjugated antibodies to diffuse into the microwells.

17. The method of claim 10, further comprising culturing the cells in the microwells, the microwells being coated in fibronectin.

18. The method of claim 10, further comprising:
extracting cells from the microwells by:
inverting the microwells,
applying a magnetic force to the microwells, the magnetic force configured to pull the cells from the microwells into the channel, and
flushing the cells from the channel with the fluid medium.

19. The method of claim 10, wherein at least 95% of the cells that are labeled with magnetic particles are pulled into the microwells subsequently extracted from the microwells.

20. A method of forming a microfluidic device, the method comprising:
obtaining a first layer of polydimethylsiloxane (PDMS);
forming a channel in the first layer of PDMS;
obtaining a second layer of polydimethylsiloxane (PDMS);
forming a plurality of microwells in the second layer of PDMS by photolithography, the plurality of microwells having a well diameter and a well depth configured to prevent fluid flow of a fluid medium into the plurality of microwells during circulation of the fluid medium in the channel layer;
bonding the first layer of PDMS to the second layer of PDMS; and
forming fluid connections between the channel and the plurality of microwells.

21. (canceled)
